(19) United States
(12) Patent Application Publication
(10) Pub. No.: US 2007/0135665 A1
(43) Pub. Date: Jun. 14, 2007

(54) Method for the production of olefins comprising 8 to 12 carbon atoms

(76) Inventors: Klaus-Diether Wiese, Haltern am See (DE); Alfred Kaizik, Marl (DE); Dietrich Maschmeyer, Recklinghausen (DE); Wilfried Buschken, Haltern am See (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/588,762

(22) PCT Filed: Dec. 23, 2004
(86) PCT No.: PCT/EP04/53693

§ 371 Date: Jan. 10, 2007

(30) Foreign Application Priority Data
Feb. 14, 2004 (DE) 10 2004 007 289.2
Jul. 8, 2004 (DE) 10 2004 033 410.2

(51) Int. Cl. C07C 1/00; C07C 2/00; C07C 11/00; C07C 29/14

(52) U.S. Cl. 585/16; 585/327; 568/882

(57) ABSTRACT

The present invention relates to the preparation of olefins or olefin mixtures having from 8 to 12 carbon atoms from one or more olefin(s) having from 4 to 6 carbon atoms by means of a four-stage synthesis. The four-stage synthesis comprises the steps of hydroformylation to form the aldehyde, hydrogenation to form the alcohol, elimination of water to form the 1-olefin and metathesis. The C₈-C₁₂-olefins obtained can be used, for example, for peparing plasticizer alcohols, in particular isononanol.

The present invention relates to the preparation of olefins or olefin mixtures having from 8 to 12 carbon atoms from one or more olefin(s) having from 4 to 6 carbon atoms by means of a four-stage synthesis.

Olefins having from 8 to 12 carbon atoms are sought-after starting materials in the chemical industry. They can, for example, be hydroformylated to produce aldehydes having one more carbon atom, which can in turn be used for important industrial products. An example of the use of aldehydes is hydrogenation of the aldehydes to the alcohols and reaction of the latter with carboxylic acids to form esters. Thus, for example, esterification of C₉-alcohols by means of phthalic anhydride leads to diisononyl phthalates which are very sought-after plasticizers in the plastics processing industry. Another industrially important process employed is oxidation of the aldehydes to the corresponding carboxylic acids which can, inter alia, be converted into oil-soluble metal salts. These are used, for example, as drying accelerators for surface coatings (siccatives) or stabilizers for PVC. Furthermore, alcohols, in particular C₁₃-alcohols, serve as precursors for surfactants.

A further example of an industrial use is the reaction of olefins with carbon monoxide and water in the presence of strong acids as catalysts to give the carboxylic acids having one more carbon atom, which is described in the literature as the KOCH reaction. This process gives tertiary branched carboxylic acid mixtures which, owing to their branched nature, are in turn very well-suited to the preparation of the abovementioned metal salts. A particularly important use of tertiary carboxylic acids is reaction with acetylene to form vinyl esters which serve as comonomers for the internal plasticization of polymers. Copolymers of vinyl esters of tertiary carboxylic acids with vinyl acetate are, for example, the basis of water-dispersible environmentally friendly paints and varnishes and energy-conserving heat-protection renders on buildings.

Among the abovementioned uses of C₈-C₁₂-olefins, their use for preparing plasticizer alcohols is the most important.

Olefins having from 8 to 12 carbon atoms can be obtained by oligomerization of ethylene, propylene or mixtures thereof. The primary product formed is a mixture of olefins having different numbers of carbon atoms. The desired olefins can be separated off from these mixtures. The unwanted olefins having a lower or higher number of carbon atoms can be converted into the target olefins only by means of multistage syntheses. The high price of ethylene and/or propylene is a disadvantage in the preparation of olefins having from 8 to 12 carbon atoms by this route.

Olefins having from 8 to 12 carbon atoms can also be obtained by dimerization of olefins or olefin mixtures having from 4 to 6 carbon atoms. The dimerization of C₄-olefins to form mixtures of isomeric octenes is of particularly great industrial importance.

There are essentially three processes, which differ in the catalyst systems used, for the industrial dimerization of olefins having from 4 to 6 carbon atoms. The oligomerization over acid catalysts (process A), in which, for example, zeolites or phosphoric acid on supports are used industrially, has been known for a long time. This gives essentially isomer mixtures of multiply branched olefins (WO 92/13818). Another process which is likewise practiced worldwide is oligomerization using soluble Ni complexes, known as the DIMERSOL process (process B) (B. Cornils, W. A. Herrmann, Applied Homogenous Catalysis with Organometallic Compounds, pages 261-263, Verlag Chemie 1996). Finally, mention must also be made of oligomerization over fixed-bed nickel catalysts, e.g. the process of OXENO Olefinchemie GmbH. The process is described in the literature as the OCTOL process (process C) (Hydrocarbon Process., Int. Ed. (1986) 65 (2. Sect. 1), pages 31-33; J. Schulze, C₄-Hydrocarbons and Derivates, Springer Verlag, Berlin 1989, pages 69-70).

Even when a pure olefin or an olefin mixture in which the individual olefins differ only in the position of the double bond and their configuration is used as starting material, the dimerization does not result in a pure substance but instead in a mixture of many structural isomers which are in turn made up of virtually all double bond isomers in various proportions, with many double bond isomers additionally displaying cis/trans isomerism. These constitutional and configurational isomers can, depending on the production process, be present in various proportions.

If, for example, dibutene is prepared from raffinate II (C₄-hydrocarbon fraction without butadiene and isobutene) or raffinate III (raffinate II after complete or partial removal of 1-butene), olefin mixtures of essentially unbranched, singly branched and doubly branched basic structures are obtained. The following data serve purely as a guide, since varying proportions of the individual structural groups are obtained depending on process conditions.

A measure of the degree of branching is the iso index. It is defined as the number of branches per molecule. Accordingly, linear octenes (n-octenes) have an iso index of 0, methylheptenes have an iso index of 1 and dimethylhexenes have an iso index of 2. In calculating the iso index of mixtures, the mass fractions of the individual groups of compounds need to be taken into account.

TABLE 1
Composition of the dimerization mixtures (dibutenes) starting
from raffinate III depending on the process employed

| | Process A | Process B | Process C |
|---|---|---|---|
| | Zeolite catalysis | Dimersol | Octol |
| n-Octenes | ˜0% | ˜6% | ˜13% |
| 3-Methylheptenes | ˜5% | ˜59% | ˜62% |
| 3,4-Dimethylhexenes | ˜70% | ˜34% | ˜24% |
| Other C₈-olefins | ˜25% | ˜1% | ˜1% |
| Iso index | >1.9 | ≅1.29 | ≅1.12 |

If other C₄ fractions which still contain isobutene, e.g. raffinate I, are used in place of raffinate II or raffinate III, the products formed also include many further, even more highly branched structures, mainly trimethylpentenes such as 2,2,4-trimethylpentenes, 2,2,3-trimethylpentenes, 2,3,4-trirnethylpentenes, 2,3,3-trimethylpentenes and other more obtained. Such dibutenes having an iso index of greater 2 are also known as “codibutylene”.

The use properties of the downstream products prepared, for example, from dibutene are often dependent on the composition and especially the degree of branching of the olefin used. This can take on quite extreme forms, as can be seen from the following examples.

An important field of use of dibutenes is the preparation of C₉-alcohols which are in turn esterified with carboxylic acids. Thus, dibutene is converted into isononanol mixtures which are esterified by means of phthalic anhydride to give isononyl phthalates which are used as plasticizers in plastics.

The degree of branching of the isononyl chains of the phthalates is closely linked to the degree of branching of the olefin used, so that the structure of the olefin mixture used is an important factor in determining the properties of the phthalates.

TABLE 2
Comparison of typical dynamic viscosities of industrially used nonyl
phthalates, where Co-HD: classical cobalt high-pressure process, 200-300
bar, 140-180° C. Rh-HD: rhodium high-pressure process, 150-300
bar, 120-130° C., unmodified rhodium catalyst or rhodium catalyst
modified with triphenylphosphine oxide

| | | | Viscosities | |
|---|---|---|---|---|
| | | | of the | |
| | Oligo- | Hydro- | isononyl | |
| | merization | formylation | phthalates | |
| Raw material | process | process | (20° C.) | |
| Raffinate I | A | Co-HD | ≅165 | mPa s |
| Raffinate II or III | A | Co-HD | 116-120 | mPa s |
| Raffinate II or III | B or C | Co-HD | 70-85 | mPa s |
| Raffinate II or III | B or C | Rh-HD | 90-100 | mPa s |

(Iso)nonyl phthalates having a low viscosity, in particular a viscosity of less than 85 mPa*s, are wanted as plasticizers on the market. Nonyl phthalates having such a viscosity can be prepared only from nonanols which are obtained from C₄ fractions containing less than 5% by mass of isobutene, based on the C₄-olefin fraction, and are usually obtained by dimerization by means of process B or C.

Linear and branched olefins are present side by side in industrial C₄-C₆-hydrocarbon mixtures, especially in C₄ or C₅ fractions from olefin crackers. The removal of the branched olefins is costly and is usually possible only after derivative formation. Thus, for example, isobutene is separated off from a C₄ fraction as MTBE after reaction with methanol.

An increasingly critical view is being taken of the use of tertiary ethers, in particular MTBE, as octane number improvers in four-stroke fuels because of possible groundwater contamination; for example, the addition of MTBE to four-stroke fuels is soon to be banned in California. It cannot be ruled out that the use of tertiary ethers could be restricted in other states, too.

In order to enlarge the raw materials base for C₈-C₁₂-olefins which are particularly well-suited to the preparation of plasticizer alcohols or surfactant alcohols and to reduce the amount of coproducts whose future marketing is uncertain, it is an object of the present invention to develop a process which is able also to utilize the branched C₄-C₆-olefins present in the industrial mixtures and/or which substantially avoids formation of high-boiling by-products, i.e. compounds having more than 12 carbon atoms, and/or which is simple to carry out.

It has now surprisingly been found that the conversion of one or more linear and/or branched olefin(s) into one or more olefin(s) having numbers of carbon atoms corresponding to the sum of the numbers of carbon atoms of two identical or different olefins present in the starting mixture can be achieved by a simple multistage synthesis comprising the following reaction steps:
a) hydroformylation of the starting olefin or starting olefins,
b) hydrogenation of the aldehyde/aldehydes formed in reaction step a) to give the corresponding alcohol(s),
c) elimination of water from the alcohol(s) formed in process step b) to give the corresponding 1-olefin(s) and
d) metathesis of the 1-olefin/1-olefins formed in step c) to eliminate ethylene and form one or more olefin(s) having numbers of carbon atoms corresponding to the sum of the number of carbon atoms of two identical or different olefins reacting with one another less two.

The present invention accordingly provides a process for preparing one or more olefin(s) having from 8 to 12 carbon atoms from one or more olefin(s) having from 4 to 6 carbon atoms by means of a four-stage synthesis, which comprises
a) hydroformylating the starting olefin or starting olefin mixture in the first reaction step,
b) hydrogenating the aldehyde(s) obtained in the first reaction step a) to form the corresponding alcohol(s),
c) preparing one or more 1-olefin(s) by elimination of water from the alcohol(s) obtained in the second reaction step and
d) obtaining one or more olefin(s) by metathesis with elimination of ethylene from the 1-olefin(s) formed in the third reaction step.

The present invention likewise provides a mixture comprising at least one olefin having from 8 to 12 carbon atoms and prepared by the process of the invention and also provides for the use of the mixture for preparing alcohols and/or aldehydes, in particular for preparing plasticizer alcohols and especially for preparing isononanol.

The advantage of the process of the invention is that both linear and branched olefins having from 4 to 6 carbon atoms or mixtures thereof can be used for preparing olefins having from 8 to 12 carbon atoms. The target olefins prepared by the process of the invention can be terminally hydroformylated and after hydrogenation of the aldehydes give primary alcohols having from 9 to 13 carbon atoms which are suitable, in particular, for the preparation of plasticizers and/or surfactants. Since, in contrast to conventional processes, branched olefins or mixtures comprising branched olefins can also be used in the process of the invention for preparing C₈-C₁₂-olefins having internal double bonds, the raw materials base for the inexpensive preparation of corresponding alcohols having from 9 to 13 carbon atoms is enlarged. These alcohols can be used as plasticizer alcohols, e.g. for the preparation of phthalates which have a low viscosity.

The process of the invention thus makes it unnecessary to separate branched olefins, e.g. isobutene, from the olefin mixture, e.g. as MTBE. In this way, the dependence of the economics of the process on a specific coproduct is avoided. In the case of the preparation of C₈-olefins from C₄-olefins, the preparation of a coproduct can, as demonstrated below, be avoided entirely.

A further advantage of the process of the invention for preparing olefins having from 8 to 12 carbon atoms from olefins having from 4 to 6 carbon atoms is that, in contrast to a process comprising an olefin oligomerization step (dimerization or trimerization step) which leads to the formation of by-products having higher numbers of carbon atoms, e.g. trimers or oligomers, such unwanted by-products are not formed.

In addition, the process of the invention makes it possible to use, depending on the available raw materials, 1-butene, isobutene or 2-butenes either alone or as a mixture comprising one or more of these compounds as starting material in the preparation of C₈-olefins which are processed to give plasticizer alcohols. The process of the invention thus increases both the raw materials base and the flexibility.

The invention makes use of processes known per se for hydroformylation, hydrogenation, elimination of water and metathesis. The concatenation according to the invention of the individual reaction steps to give a process for preparing olefins having from 8 to 12 carbon atoms from olefins having from 4 to 6 carbon atoms achieves the abovementioned advantages.

The process of the invention and the products prepared using it are described by way of example below, without the invention being restricted to these illustrative embodiments. If ranges, general formulae or classes of compounds are referred to below, these are intended to encompass not only the respective ranges or groups of compounds which are explicitly mentioned but also all part ranges and subgroups of compounds which can be obtained by taking out individual values (ranges) or compounds. To give a better understanding of the process of the invention, the process steps a) to d) are additionally illustrated by a description of a treatment according to the invention of a hydrocarbon stream comprising C₄-olefins, without the invention being restricted thereto.

The process of the invention for preparing at least one olefin having from 8 to 12 carbon atoms from at least one olefin having from 4 to 6 carbon atoms by means of a four-stage synthesis comprises
a) hydroformylating the at least one starting olefin in the first process step,
b) hydrogenating the at least one aldehyde obtained in the first step a) in a second process step to form the corresponding alcohol,
c) preparing at least one 1-olefin by elimination of water from the at least one alcohol obtained in the second process step b) and d) obtaining at least one olefin by metathesis with elimination of ethylene of the at least one 1-olefin(s) obtained in the third process step c).

The process of the invention can be carried out using only one olefin or else a mixture of olefins. In particular, a mixture of olefins having from 4 to 6 carbon atoms can be used and a mixture of olefins having from 8 to 12 carbon atoms can accordingly be obtained. If “an olefin” is referred to in the context of the present invention, this is intended to mean not only a single molecule but a group, e.g. a stream, of olefinically unsaturated compounds which have the same empirical formula and optionally have one and the same structure. If olefins are referred to in the context of the present invention, this means groups/streams of olefinically unsaturated compounds which have different numbers of carbon atoms and/or different structures.

In particular, the process of the invention is carried out using hydrocarbon mixtures comprising monoolefins having from 4 to 6 carbon atoms which are, however, preferably virtually free of polyunsaturated hydrocarbons such as dienes or acetylene derivatives. The process of the invention can be carried out using hydrocarbon mixtures comprising olefins having three different numbers of carbon atoms, mixtures comprising olefins having two different numbers of carbon atoms or mixtures comprising olefins having the same number of carbon atoms.

The olefins themselves or the olefin-containing hydrocarbon mixtures which can be used in the process of the invention can originate from a variety of sources. Olefins having 6 carbon atoms are, for example, present in low-boiling fractions obtained in the processing of petroleum. A source of olefins having 5 carbon atoms is the C₅ fraction from olefin crackers.

Preference is given to using hydrocarbons having 4 carbon atoms in the process of the invention. In particular, it is possible to use industrial C₄ fractions which have an isobutene content of greater than 3% by weight, preferably greater than 10% by weight and particularly preferably greater than 20% by weight. The most important source of C₄-olefins is the C₄ fraction from steam crackers. The butadiene is removed therefrom by extraction or extractive distillation or by selective hydrogenation to a mixture of n-butenes to produce a hydrocarbon mixture (raffinate I or hydrogenated C₄ fraction) comprising isobutene, 1-butene and the two 2-butenes. Another raw material for C₄-olefins is the C₄ fraction from FCC plants, which can be worked up as described above. C₄-Olefins prepared by the Fischer-Tropsch synthesis are likewise a suitable starting material after selective hydrogenation of the butadiene present therein to n-butenes. Furthermore, olefin mixtures obtained by dehydrogenation of C₄-hydrocarbons or by means of metathesis reactions or other industrial olefin streams can also be suitable starting materials.

Owing to the frequently high cost of separation, the olefins present in the industrial mixture used as starting mixture are generally not separated, but instead the mixtures are used directly in process step a).

Process Step a)

The hydroformylation of all olefins in the reaction mixture can be carried out in one step. This requires a catalyst which is able to hydroformylate olefins having different positions of the double bond and/or different numbers of branches. However, catalysts which are suitable for this purpose usually give only a low selectivity for the formation of products (aldehydes, alcohols, formates) resulting from terminal hydroformylation and/or display a reaction rate which is too low for an industrial process.

If the end products (olefins having from 8 to 12 carbon atoms) are to be used mainly as precursors for plasticizer alcohols and surfactant alcohols, it is advantageous to carry out the hydroformylation so that a high proportion of products formed by terminal hydroformylation is obtained, since only the terminally hydroformulated products have the same degree of branching as the corresponding starting olefins, while internal hydroformylation results in the degree of branching of the product formed being increased by 1.

The olefins present in an industrial mixture have considerably different reactivities in the hydroformylation. In general, olefins having terminal double bonds are more reactive than olefins having internal double bonds and linear olefins are more reactive than branched olefins.

In the specific case of C₄-olefins, 1-butene is more reactive than isobutene and isobutene is more reactive than the two 2-butenes. This differing reactivity can be utilized to obtain a high proportion of products which have been formed by terminal hydroformylation, i.e. 1-butene should be converted mainly into valeraldehyde and not 2-methylbutanal, isobutene should be converted into 3-methylbutanal and not 2,2-dimethylpropanal and the two 2-butenes should if possible be converted into a large proportion of valeraldehyde (pentanal) and little 2-methyl-butanal.

Since there is as yet no catalyst which simultaneously effects the conversion both of 1-butene and of isobutene and the 2-butenes into products formed by terminal hydroformylation at a satisfactory rate, the hydroformylation is preferably carried out in at least two stages.

In a first stage, the hydroformylation is carried out using a suitable catalyst under conditions under which only α-olefins (1-butene, isobutene) but not the 2-butenes are converted into the corresponding aldehydes. In this stage, the conditions are selected so that 1-butene is converted very selectively into valeraldehyde and isobutene is converted very selectively into 3-methyl-butanal. Catalysts used are, for example, compounds comprising rhodium and triorganic phosphorus compounds, in particular phosphines, as ligands. The reaction can be carried out in a homogeneous phase (analogous to the UCC process EP 0 562 451) or in a heterogeneous phase (analogous to the Rhone-Poulenc-Ruhrchemie process DE 026 27 354, EP 0 562 451). Owing to the greater ease with which the catalyst can be separated off, this first stage of the process step a) is preferably carried out by the second method. The reaction temperatures are preferably from 70 to 150° C., more preferably from 100 to 130° C. The process pressures are preferably from 2 to 20 MPa, more preferably from 3 to 6 MPa.

The hydroformylation of the 1-olefins can optionally be carried out in a multiphase system in which the starting material, product and synthesis gas are dispersed in a continuous catalyst phase at high superficial velocities. Such processes are described, for example, in DE 199 25 384 A1 and DE 199 57528 A1, which are hereby expressly incorporated by reference.

The hydroformylation of the 1-olefins can be carried out in one or two stages. In the two-stage hydroformylation, predominantly 1-butene is reacted in the first reactor and mainly isobutene is reacted in the second reactor. The same catalysts or different catalysts can be used in the two reactors. When the same catalysts are used, joint work-up of the catalyst is possible.

After the hydroformylation of 1-butene and part of the isobutene in the first stage of process step a) as just described, the 2-butenes and possibly isobutene and at most traces of 1-butene remain in the starting hydrocarbon mixture. This mixture can be hydroformylated as such using a different catalyst system or can be separated into two fractions of which comprises isobutene and the other comprises the two 2-butenes before being hydroformylated. Preference is given to fractionating the mixture and hydroformylating the isobutene-containing fraction and the 2-butene-containing fraction separately.

The fraction comprising mainly 2-butenes as olefins is optionally oligomerized to form predominantly C₈-olefins. This is carried out with the aid of nickel-containing catalysts, preferably fixed-bed catalysts, as in, for example, the OCTOL process (see above).

The isobutene or the isobutene-containing fraction can be hydroformylated with high selectivity to 3-methylbutanal. Suitable catalysts for this purpose are rhodium complexes containing monodentate or polydentate phosphite ligands. Such monodentate phosphite ligands are, for example, triaryl phosphites whose aryl groups have a bulky group in the ortho position relative to the phosphite oxygen and are also substituted in the m or p position, e.g. tris(2,4-di-tert-butylphenyl) phosphite. The hydroformylation of isobutene using a catalyst system comprising rhodium and a bisphosphite is described, for example, in U.S. Pat. No. 4,668,651, U.S. Pat. No. 4,769,498 and WO 85/03702, whose disclosure is hereby expressly incorporated by reference into the present description.

The isobutene fraction which has been separated off can optionally be partly recirculated to the preceding hydroformylation stages. If the saturated hydrocarbons are separated off from the isobutene, it can be recirculated in its entirety.

The hydroformylation of 2-butenes or 2-butene-containing fractions can be carried out with the aid of the various catalysts and usually forms a mixture of 2-methylbutanal and valeraldehyde. In most cases, 2-methylbutanal is the main product. The use of unmodified cobalt catalysts as catalysts for the hydroformylation of 2-butenes is described in EP 0 646 563 and the use of unmodified rhodium is described in EP 0 562 451. Furthermore, the hydroformylation of 2-butenes can be carried out using the same catalyst system used for the hydroformylation of isobutene, namely a complex of rhodium and a monodentate triaryl phosphite. High selectivities to valeraldehyde can be obtained when using a catalyst comprising rhodium and bulky aromatic bisphosphites as described, for example, in EP 0 213 639. However, the reaction rates are low for an industrial process.

As indicated above, the olefins present in the starting material can be hydroformylated separately or jointly. When the linearity of the end products is not of great importance, it is advantageous to hydroformylate the olefins jointly. On the other hand, if an end product having very little branching is desired, the hydroformylation has to be carried out in at least two stages. In the case of a C₄-olefin mixture, the latter case means that 1-butene and possibly isobutene is/are reacted in the first reactor and the remaining olefins are reacted in the downstream reactor(s).

The catalyst is separated off from the hydroformylation mixtures by known methods. For example, in the case of processes in which the rhodium catalyst is present in a homogeneous reaction mixture, the catalyst can be separated off by distillation. In the case of a reaction in a heterogeneous phase (two liquid phases), the catalyst is separated off by phase separation (Ed. B. Cornils, W. A. Herrmann, Applied Homogeneous Catalysis with Organic Compounds, Vol. 1, p. 80, VCH-Verlag, 1996).

After catalyst removal, the hydroformylation mixtures can either be used directly in process step b) or can be separated into two or more fractions by distillation or by means of other separation methods. In particular, it can be advantageous to work up the hydroformylation mixture so as to give one or more fractions consisting essentially of aldehydes.

Process Step b)

The hydroformylation mixtures after catalyst removal or the aldehydes or aldehyde-containing fractions separated off from these by means of a separation process such as distillation are hydrogenated in the second process step b) of the process of the invention. Here, the hydroformylation mixtures can be hydrogenated separately or jointly. The hydrogenation converts the aldehydes into the corresponding saturated alcohols. These are, for example, n-pentanol, 2-methylbutanol and 3-methylbutanol.

For the hydrogenation, it is possible to use, for example, nickel, copper, copper/nickel, copper/chromium, copper/chromium/nickel, zinc/chromium, nickel/molybdenum catalysts. The catalysts can be unsupported or the hydrogenation-active materials or their precursors can have been applied to supports such as silicon dioxide or aluminum oxide. Preferred catalysts for hydrogenating the hydroformylation mixtures comprise, in each case, from 0.3 to 15% by mass of copper and nickel, and, as activators, from 0.05 to 3.5% by mass of chromium and advantageously from 0.01 to 1.6% by mass, preferably from 0.02 to 1.2% by mass, of an alkali component on a support material, preferably aluminum oxide or silicon dioxide. The amounts indicated are based on the not-yet-reduced catalyst. The alkali component is optional. The catalysts are advantageously used in a form in which they offer little resistance to flow, e.g. in the form of granules, pellets or shaped bodies such as tablets, cylinders, extrudates or rings. They are advantageously activated before use, e.g. by heating in a stream of hydrogen.

The hydrogenation, preferably a liquid-phase hydrogenation, is generally carried out at a total pressure of from 0.5 to 50 MPa, in particular from 1.5 to 10 MPa. A hydrogenation in the gas phase can also be carried out at lower pressures, in which case correspondingly large volumes of gas are then present. If a plurality of hydrogenation reactors are employed, the total pressures in the individual reactors can be identical or different within the pressure limits mentioned. The reaction temperatures in the hydrogenation in the liquid or gaseous phase can generally be from 120 to 220° C., in particular from 140 to 180° C. Such hydrogenations are described, for example, in the patent applications DE 198 42 369 and DE 198 42 370, which are hereby expressly incorporated by reference.

In the process of the invention, the hydrogenation is preferably carried out in the presence of water. The water required can be present in the feed to the reactor. However, it is also possible for water to be fed into the hydrogenation apparatus at a suitable point. In the case of a gas-phase hydrogenation, water is advantageously introduced in the form of water vapor. A preferred hydrogenation process is liquid-phase hydrogenation with addition of water, as described, for example, in DE 100 62 448. The hydrogenation is particularly preferably carried out at a water content of from 0.05 to 10% by mass, in particular from 0.5 to 5% by mass, very particularly preferably from 1 to 2.5% by mass. The water content is determined in the output from the hydrogenation.

The mixtures obtained from the hydrogenation can either be used directly in process step c) or else are separated into two or more fractions by distillation or by means of other separation methods. In particular, it can be advantageous to work up the hydrogenation mixture so that one or more fractions consisting essentially of alcohols having the same number of carbon atoms are obtained.

When part of the linear olefins present in a C₄-hydrocarbon fraction is hydroformylated internally, it can be advantageous to separate off all or part of the 2-methylbutanol formed therefrom by hydrogenation.

Process Step c)

In the third process step c), the corresponding 1-olefins are prepared by elimination of water from the alcohol mixture or the alcohol-containing fractions obtained after the hydrogenation in step b). In the process of the invention, this dehydration is carried out continuously or batchwise in the gas phase or mixed liquid/gas phase over suspended catalysts or particulate catalysts located in a fixed bed. Owing to the ease with which the reaction products can be separated from the reaction mixture, the elimination of water is preferably carried out in the gas phase or the mixed gas/liquid phase over solid catalysts in the temperature range from 200 to 500° C. Preference is given to carrying out a continuous dehydration over a fixed-bed catalyst. As catalysts, it is possible to use oxides of the alkaline earth metals, of aluminum, indium, gallium, of silicon, scandium, yttrium, lanthanum, titanium, zirconium, thorium and the rare earths. It is also possible to use mixed oxides and combinations of the above oxides. In the case of some catalysts, a particular acidity can be set by addition of alkali metal oxides.

The specialist scientific literature discloses, for example, the following suitable catalysts:
NiO/Al₂O₃; CuO/Al₂O₃; Al₂O₃ (J. Mol. Catal. A. Chem. (1997), 121 (2-3), pp. 157-159);
ZrO₂; sulfated ZrO₂ (J. Mol. Cat. A. Chem (1997), 118 (1), pp. 88-89);
Al₂O₃; Co₂O₃; ThO₂; In₂O₃ (J. Catal. (1988), 110 (2), pp. 416-418);
HfO₂/ZrO₂ (J. Phys. Chem. (1980), 84 (1), 55-56);
Al₂O₃/Na₂O; ThO2 (J. Catal. (1981), 68 (2), pp. 383-387);
ThO₂ (J. Org. Chem. (1967), 32 (11), 3386-3389);
La₂O₃ (Z. Phys. Chem.(1985), 144, pp. 157-163);
Ga₂O₃ (J. Org. Chem. (1977), 44 (13), pp. 2142-2145);
ThO₂; Al₂O₃ (J. Org. Chem. (1972), 37 (8), pp. 1240-1244).

The catalysts and the reaction conditions are preferably selected so that the formation of by-products such as ethers and also isomerization of the 1-olefins formed to olefins having internal double bonds are largely avoided. For this reason, preference is given to using basic or strongly basic catalysts for the preparation of 1-olefins from the primary alcohols by elimination of water in process step c) of the process of the invention. The catalysts used can comprise aluminum oxide (Al₂O₃) and/or zirconium oxide (ZrO₂) as main components together with alkali metal oxides and/or alkaline earth metal oxides. Further components which may be present in the catalyst are titanium dioxide, silicon dioxide and/or thorium oxide in an amount of from 0.01 to 3% by mass, preferably from 0.5 to 5% by mass.

The proportion of basic metal oxides (hydroxides are calculated as oxides) in the catalyst is preferably from 0.01 to 10% by mass, particularly preferably from 0.1 to 5% by mass, very particularly preferably from 0.1 to 3% by mass. Preferred alkali metal oxides are sodium oxide and/or potassium oxide. As alkaline earth metal oxides, preference is given to using magnesium oxide, strontium oxide and/or barium oxide. The catalyst used in step c) is very particularly preferably a y-aluminum oxide modified with barium oxide (BaO), which formally consists of barium oxide and aluminum oxide.

Preference is given to using γ-aluminum oxides having a BET surface area of from 80 to 350 m²/g, preferably from 120 to 250 m²/g (determined by N₂ adsorption in accordance with DIN 66131). The catalysts are produced by known methods. Customary methods are, for example, precipitation, impregnation or spraying of an Al₂O₃ body with an appropriate salt solution and subsequent calcination.

It can likewise be advantageous to use catalysts as are described in DE 103 59 628 and comprise from 80 to 99 parts by mass of zirconium dioxide, from 0.5 to 10 parts by mass of yttrium oxide and from 0.1 to 3 parts by mass of alkaline earth metal oxides or alkali metal oxides.

The catalysts are preferably used in the form of spheres, tables, cylinders, extrudates or rings.

In the continuous elimination of water, it is possible to use different process variants. The process step c) can, for example, be carried out adiabatically, polytropically or virtually isothermally, i.e. with a temperature difference of typically less than 10° C. The process step can be carried out in one or more stages. In the latter, case all reactors, advantageously tube reactors, can be operated adiabatically or virtually isothermally. It is likewise possible to operate one or more reactors adiabatically and operate the others virtually isothermally. The elimination of water is preferably carried out in a single pass. However, it can also be carried out with recirculation of product. In single-pass operation, the WHSV over the catalyst is from 0.01 to 30 kg, preferably from 0.1 to 10 kg, of alcohol per kg of catalyst and per hour. In the elimination of water in process step c), the temperature in the catalyst bed is preferably from 200 to 450° C., in particular from 250 to 350° C. The elimination of water (dehydration) can be carried out under reduced pressure, under superatmospheric pressure or at atmospheric pressure.

The feed to process step c) can be introduced into the dehydration reactor in pure form or in diluted form. As diluents, it is possible to use gases or gas mixtures, for example nitrogen, hydrogen,carbon monoxide, carbon dioxide, synthesis gas, methane or steam, or organic solvents which are inert under the reaction conditions and can easily be separated off from the output from the reaction.

To achieve a very high selectivity to 1-olefin formation, it has been found to be advantageous to react only part of the alcohol introduced. Process step c) is preferably carried out so that the conversion in a single pass is from 30 to 90%.

The product obtained from the process step c) is a mixture comprising at least one 1-olefin. The reaction mixture is preferably separated into fractions comprising the 1-olefin(s) formed, the primary alcohol(s) and possibly by-products, for example ethers or carbonyl compounds such as aidehydes. The separation can be carried out, for example, by distillation. The olefin fraction obtained after the separation can optionally be worked up to give pure 1-olefin. The alcohol-containing fraction, which comprises the unreacted alcohol, is preferably recirculated to the dehydration. The aldehyde (for example n-pentanal) formed as by-product in, for example, the dehydration of 1-hydroxyalkane (1-pentanol) can be reused after hydrogenation to the corresponding alcohol and thus does not constitute a loss of material. The hydrogenation can be carried out in a separate hydrogenation apparatus, but it can be advantageous to feed the aldehydes obtained as by-products into the hydrogenation (step b) preceding the dehydration. As a result of this separation of the aldehydes from the mixture obtained in the dehydration, subsequent hydrogenation and recirculation of the alcohols obtained to the dehydration, olefins can be obtained in a particularly high selectivity in the dehydration according to the invention.

The fraction comprising at least one 1-olefin can optionally be fractionated. The individual fractions can be used separately in the next step of the synthesis. It is also possible for one or more fraction(s) not to be used in the next step of the synthesis but instead to be used in another way. It is also possible to prepare a mixture of two or more of the fractions and to use this in the next process step d). In this way, it is possible to obtain olefin mixtures having very different iso indices as products of process step d).

If the starting material used in process step a) of the process of the invention is, for example, a hydrocarbon stream, in particular a C₄-hydrocarbon mixture comprising or consisting of isobutene and linear butenes, e.g. raffinate I or selectively hydrogenated C₄ fraction from a cracker, hydroformylation, hydrogenation and elimination of water (process steps a) to c)) give a C₅-olefin mixture comprising the 1-olefins 1-pentene, 3-methyl-1-butene and possibly 2-methyl-1-butene. 1-Pentene has a boiling point of 30° C. at atmospheric pressure, 2-methyl-1-butene has a boiling point of 31.2° C. and 3-methyl-1-butene has a boiling point of 20.1° C. Owing to the significantly different boiling points, 3-methyl-1-butene can, after process step c), easily be separated off by distillation from the other two isomers present in the 1-olefin fraction comprising olefins having five carbon atoms which is obtained. 3-Methyl-1-butene is an olefin for which there are a number of industrial uses. For example, it is used for the modification of polypropylene.

The removal of part or all of the 3-methyl-1-butene reduces the degree of branching in the remaining isomer mixture. The target product (C₈-olefins) formed in the next step of the synthesis therefore has a lower degree of branching (iso index). This is particularly advantageous when the target product is used as precursor for plasticizer alcohols.

The product which can be obtained from the process steps a) to c) when using 1-butene, isobutene and/or 2-butenes can be seen in Table 3.

TABLE 3
Products of process steps a) to c)

| | Aldehyde | Alcohol | Olefin |
|---|---|---|---|
| | (products of | (products of | (products of |
| C4 | process | process | process |
| component | step a)) | step b)) | step c)) |
| 1-Butene | Pentanal + | Pentanol + | 1-Penten + |
| | 2-methyl- | 2-methyl- | 2-methyl- |
| | butanal | butanol | 1-butene |
| 2-Butene | 2-Methyl- | 2-Methyl- | 2-Methyl- |
| | butanal + | butanol + | 1-butene + |
| | pentanal | pentanol | 1-pentene |
| Isobutene | 3-Methylbutanal | 3-Methylbutanol | 3-Methyl- |
| | (+2,2- | (+2,2- | 1-butene |
| | dimethylpropanal) | dimethylpropanol) | |

Process Step d)

The product obtained from process step c) can be transferred wholly or partly to process step d). The olefin(s) obtained in process step c) is/are preferably subjected to metathesis in process step d) under conditions under which virtually no isomerization of the starting materials and/or the products occurs as a result of a shift of a double bond. In this step, ethylene is eliminated and one or more olefin(s) having a central double bond is/are formed. The number of carbon atoms in the olefins formed is the sum of those of the two 1-olefins reacting with one another less two. If a mixture of 1-olefins having the same number n of carbon atoms is used, the product olefins have 2n−2 carbon atoms. For example, if mixtures comprising olefins having n and m carbon atoms are used, the product olefins formed have 2n−2, 2m−2 and n+m-2 carbon atoms. The maximum number of olefins present in the product mixture is, disregarding configurational isomers, given by the sum of the various linear combinations. For example, a 1-olefin mixture comprising the C₅-olefins, 1-pentene and 3-methyl-1-butene can be converted into the following C₈-olefins:

4-octene (cis and trans), 2-methyl-3-heptene (cis and trans) and 2,5-dimethyl-3-hexene (cis and trans).

To obtain exclusively linear C₈-olefins, it is necessary for the feed stream to be freed of all olefins with the exception of 1-pentene. Such a removal can be carried out, for example, by distillation. If exclusively C₈-olefins are the objective of process step d), the starting mixture must contain only olefins which have 5 carbon atoms and whose double bond is terminal. To prevent internal olefins from being formed from the terminal olefins by isomerization, the metathesis reaction is preferably carried out under conditions under which isomerization is very largely avoided. Since 2-methyl-1-butene frequently does not undergo a metathesis reaction with itself but can be isomerized under metathesis conditions firstly to 2-methyl-2-butene and further to 3-methyl-1-butene, the use of hydrocarbon mixtures comprising exclusively 2-methyl-1-butene in process step d) can result in cross-metathesis and homometathesis of the 2-methyl-2-butene being formed with itself and with 2-methyl-1-butene or 3-methyl-1-butene to form many different compounds, in particular 3-methyl-2-butene and isobutene. To avoid this, preference is given to feeding only streams having a ratio of 1-pentene to 2-methyl-1-butene of greater than or equal to 1:1, preferably from 1.1:1 to 100 000:1, particularly preferably from 1000:1 to 10 000:1, to process step d). Should the product stream obtained from process step c) not meet these conditions, the ratio of 1-pentene to 2-methyl-1-butene can be adjusted either by addition of 1-pentene or by removal of at least part of the 2-methyl-1-butene, e.g. by distillation.

The metathesis can be carried out continuously or batchwise in a homogeneous liquid phase or heterogeneously over a solid catalyst. The metathesis in the process of the invention is preferably carried out over a particulate catalyst arranged in a fixed bed. The reaction can be carried out over the known supported catalysts comprising, for example, oxides of tungsten, molybdenum or rhenium. In order to avoid significant isomerization of the olefins and the associated formation of by-products, rhenium catalysts are preferably used in the process of the invention.

As catalysts, it is possible to use, for example, the supported rhenium catalysts known from the literature, viz. Re₂O₇ on γ-Al₂O₃ or on mixed supports such as SiO₂/Al₂O₃, B₂O₃/SiO₂/Al₂O₃ or Fe₂O₃/Al₂O₃. The proportion of rhenium in these catalysts is from 1 to 20% by mass, preferably from 5 to 15% by mass. These catalysts can further comprise alkali metal compounds or alkaline earth metal compounds to set the acidity. Examples of the production and use of such catalysts are given, for example, in DE 014 42 597, EP 0 268 525, EP 0 282 313, EP 0 438 134, EP 0 444 264, EP 0 710 638, EP 0 710 639 (based on Re₂O₇) or DE 017 67 082, EP 0 319 065 and EP 0 245 653 (based on MoO₃), which are hereby expressly incorporated by reference.

The process step d) is preferably carried out at a temperature of from 20 to 200° C., when using a catalyst based on Re₂O₇ preferably at a temperature of from 20 to 120° C., particularly preferably at a temperature of from 50 to 100° C. The metathesis of the 1-olefins can be carried out in the liquid phase, in the gas phase or in the mixed liquid/gas phase. In the case of a reaction in flow tubes, the reaction times are preferably from 5 to 40 minutes, particularly preferably from 10 to 25 minutes. Furthermore, it is possible to carry out the metathesis of process step d) in a reactive distillation column.

The conversion in process step d) is preferably from 25 to 80%, particularly preferably from 35 to 60%. The reaction mixture obtained from process step b) is preferably worked up by distillation. Unreacted 1-olefin(s) can be recirculated to process step d). It can be advantageous for a substream of the reaction mixture obtained in process step d) to be recirculated directly to the reactor or the process step and the other substream to be worked up, e.g. by distillation. The ethylene formed can be utilized for known syntheses or, in the case of stand-alone plants, can optionally be converted into C₄-olefins which can in turn be utilized as starting material.

A specific, preferred embodiment of the present invention is a process for preparing a mixture of isomeric octenes from a C₄-hydrocarbon mixture comprising isobutene and linear butene as olefins. In this case, the olefins in the starting hydrocarbon mixture are converted by hydroformylation into the corresponding C₅-aldehydes which after hydrogenation and elimination of water give a mixture of isomeric 1-pentenes. Metathesis of these with elimination of ethylene gives the target olefins, in particular 4-octene or octene mixtures.

The process of the invention thus enables, inter alia, isobutene to be used for preparing plasticizer alcohols. In the conventional preparation of plasticizer alcohols or their starting materials, viz. C₈-olefins, the isobutene is usually firstly separated off from the C₄-olefin-containing stream, since dimerization in the presence of isobutene leads to highly branched C₈-olefins. In particular the dimerization of 2 isobutene molecules and subsequent hydroformylation and hydrogenation leads to 3,5,5-trimethylhexanol which has an iso index of 3. Plasticizers prepared therefrom have a very high viscosity, which is why they are now no longer used to any significant extent. In the process of the invention, the isobutene can be converted easily and with virtually no formation of by-products into 3-methylbutanal by hydroformylation. Hydrogenation and terminal dehydrogenation gives 3-methyl-1-butene. In the metathesis, two molecules of 3-methyl-1-butene are converted into 2,5-dimethyl-3-hexene and ethene. Hydroformylation of 2,5-dimethyl-3-hexene and subsequent hydrogenation gives 3,6-dimethylheptanol as main product. The internal product, viz. 2-isopropyl-4-methyl-pentanol, is usually formed only in small amounts. The advantage of the process of the invention can clearly be seen, since an alcohol mixture having an iso index of close to 2 is obtained. When converted into plasticizers such as di-C₉ phthalate, this gives a plasticizer whose use properties such as the viscosity are within the range of customary market products.

The iso index or the viscosity can be improved further by dimerizing the ethene formed in the metathesis to 1-butene and recirculating this as raw material to process step a). In accordance with the reaction scheme which has been comprehensively discussed, linear octenes (iso index 0) are then formed and part of the isobutene is incorporated into methylheptenes (iso index 1).

For the sake of completeness, mention will be made of a further opportunity for raw material savings. Depending on its origin, raw C₄ fraction from a cracker naturally contains varying amounts of saturated butanes, viz. n-butane and isobutane. After the complete reaction sequence, these remain as inerts and crude butane, which in contrast to the conventional procedure no longer contains any significant amount of reactive components, is obtained. Finally, certain amounts of by-products which cannot be utilized are formed, as in any industrial process. This has not been discussed before, but will be well-known to any person skilled in the art. For example, although the hydroformylation can be carried out highly selectively, small amounts of high boilers will always be formed, for example by aldolization of the aldehydes formed in the hydroformylation. The hydrogenation of the aldehydes also forms small amounts of high boilers, as does the subsequent dehydration. Although the dehydration proceeds largely terminally, for example with a selectivity of up 95%, the remainder is internal olefins. In the metathesis, these then give products having other than 2 and 8 carbon atoms, namely propene, butenes, pentenes and hexenes. If relatively large amounts of 2-methyl-1-butene are present, the amount of by-products can increase further, since 2-methyl-1-butene is, in contrast to 3-methyl-1-butene and 1-pentene, easily isomerized to the internal olefin, which leads to by-products in the metathesis.

Since the first step of the actual reaction sequence is the hydroformylation, synthesis gas, viz. a mixture of carbon monoxide and hydrogen in an approximate molar ratio of 1:1, is required. This is usually prepared by partial oxidation of hydrocarbons, for example by partial oxidation of heavy heating oil. This can be replaced by the totality of by-products of the process sequence, since only by-products containing C, H and possibly O, e.g. the high boilers from the hydroformylation, are formed. All the inert hydrocarbons, butane, isobutane and any residual unreacted olefins, namely the crude butane remaining at the end of the sequence, can also be used for generating synthesis gas. Since ethylene is ultimately formed from the synthesis gas in the metathesis step, all by-products are finally converted completely into product of value. The inert butanes and isobutane, which amount to considerable quantities in large-scale production, are also brought in this way into the value-added chain via synthesis gas without costly processes such as dehydrogenation of the butane to butenes. Finally, if the ethylene is used for producing 1-butene by dimerization, complete conversion of the entire C₄ fraction from a cracker into a single product without by-products or waste materials and thus complete utilization of the raw material is achieved.

The process of the invention makes it possible to prepare mixtures comprising at least one olefin having from 8 to 12 carbon atoms. The process of the invention makes it possible to prepare, for example, isooctene using a C₄ fraction having an isobutene content of greater than 3% by weight, preferably greater than 10% by weight, as starting material. The target olefins or mixture formed can be used as starting material for the preparation of many further materials, in particular aldehydes, alcohols and/or carboxylic acids, especially for the preparation of plasticizer alcohols. For example, a C₈-olefin mixture (isooctene) prepared according to the invention can be converted by hydroformylation and subsequent hydrogenation into a nonanol mixture (isononanol) which is suitable for the preparation of plasticizers.

The following examples illustrate the invention without restricting its scope which is defined by the description and the claims.

### EXAMPLE 1

### Hydroformylation of Raffinate I

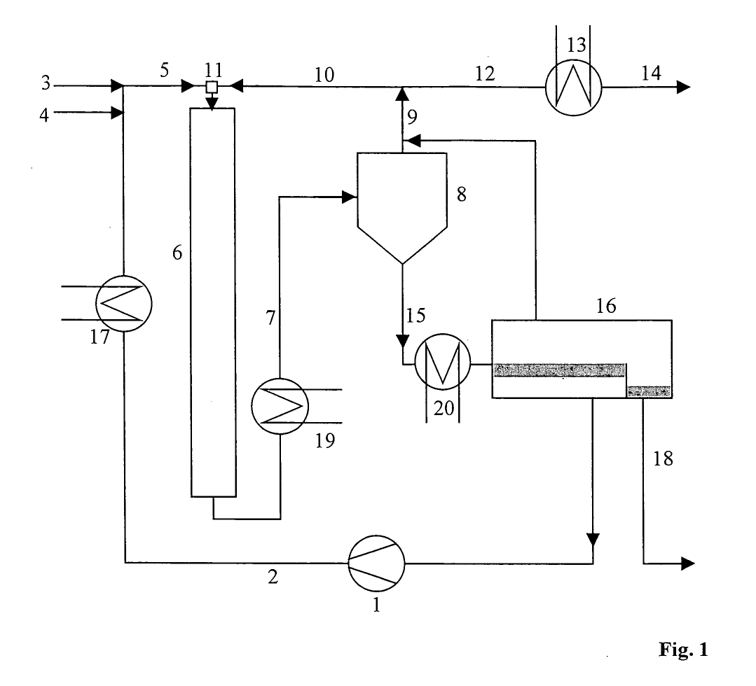

The hydroformylation was carried out in an experimental apparatus as shown schematically in FIG. 1. The reaction tube 6 had a length of 3 m and a diameter of 17.3 mm (volume=705 ml) and contained static mixing elements having a hydraulic diameter of 2 mm from Sulzer. The aqueous catalyst solution 2 was circulated by means of a pump 1. Raffinate I 3 and synthesis gas 4 were mixed into the catalyst solution. The multiphase mixture 5 obtained in this way was pumped via the mixing nozzle 11 through the tube reactor 6. The resulting mixture 7, consisting of product, unreacted starting material and the catalyst, was degassed in the container 8. The gas mixture leaving the container via line 9 was not recirculated to the reactor (line 10 closed, but optionally possible). The C₄-hydrocarbons present in the gas stream 12 were condensed in the cooler 13 in the temperature range from −50 to −78° C. 14. The liquid stream 15 obtained after degassing in the container 8 was fed into a phase separation vessel 16. Here, the aqueous catalyst phase 2 was separated off and returned to the circuit. The heat of reaction could be removed elegantly via external heat exchangers (17, 19 and 20). The organic phase 18 comprised the hydroformylated product.

The catalyst solution used consisted of the following components:
290.3 g of TPPTS (triphenylphosphine trisulfonate) in the form of the trisodium salt (0.511 mol)
403 g of ethylene glycol
318 g of water
1.88 g of rhodium acetate (0.0085 mol)

The rhodium concentration in the catalyst phase was thus 800 ppm by mass and the molar rhodium/phosphorus ratio was 1/60.

As synthesis gas, use was made of a mixture of 50% by volume of hydrogen and 50% by volume of carbon monoxide.

The raffinate I used had the following composition:

| | |
|---|---|
| Isobutane | 3.10% by mass |
| n-Butane | 7.90% by mass |
| trans-2-Butene | 8.80% by mass |
| 1-Butene | 28.10% by mass |
| Isobutene | 45.54% by mass |
| cis-2-Butene | 6.48% by mass |
| Miscellaneous | 0.08% by mass |

400 kg of catalyst solution, 600 standard 1/h of synthesis gas and 3 kg of raffinate I were passed through the tube reactor at 115° C. and a pressure of 50 bar. Separating off the catalyst phase gave, including the gas condensate, 3340 g of product phase having the following composition (in % by mass).

| | |
|---|---|
| i-Butane | 2.78 |
| n-Butane | 7.10 |
| trans-Butene | 7.90 |
| 1-Butene | 8.41 |
| Isobutene | 38.86 |
| cis-Butene | 5.82 |
| 2-Methylbutanal | 1.17 |
| 3-Methylbutanal | 3.04 |
| n-Pentanal | 24.10 |
| 2-Methylbutanol | 0.06 |
| Methylpropyldioxolane | 0.07 |
| 1-Pentanol | 0.27 |
| 2-n-Butyl-1,3-dioxolane | 0.31 |
| Miscellaneous | ca. 0.1 |
| Ethylene glycol | <0.01 |

This corresponds to a 1-butene conversion in a single pass of 66.66%. The selectivity to terminally hydroformylated products (n-pentanal, n-pentanol, 2-n-butyl-1,3-dioxolane) was 95.4%. Isobutene was converted to an extent of 5%, with the selectivity for the formation of the 3-methylbutanol formed by terminal hydroformylation being greater than 99%.

Reaction of the 2-butenes could not be detected.

Owing to the lack of a suitable pressure distillation apparatus, the fractional distillation of the reaction mixture was not carried out. In industrial production, the synthesis gas would be separated off, possibly together with part of the C₄-hydrocarbons, and recirculated to the tube reactor. The remaining mixture would be separated into a crude aldehyde mixture, an isobutane fraction (bp=−11.4° C.), a fraction comprising isobutene (bp=−6.9° C.) and 1-butene (bp=−6.3° C.) and a fraction comprising cis-2-butene (bp=3.7° C.), trans-2-butene (bp=0.9° C. and n-butane (bp=−0.5° C.).

The fraction comprising the unreacted 1-olefins (1-n-butene, isobutene) could be recirculated to the tube reactor, so that complete conversion of the 1-olefins in the tube reactor is possible. The fraction comprising the 2-butenes could be used in a second hydroformylation stage (see Example 2). The dioxolanes could be separated off from the crude aldehyde by distillation and be introduced into the tube reactor. In the presence of the aqueous catalyst solution, these are in equilibrium with the parent aldehydes. The separation of such mixtures by distillation is prior art.

This example shows that the two α-olefins (isobutene and 1-butene) are converted in selectivities of over 95% into products formed by terminal hydroformylation without reaction of the 2-butenes present in the mixture and that complete conversion is possible when the unreacted α-olefins are recirculated.

### EXAMPLE 2

### Hydroformylation of 2-butenes

To prepare a hydroformylation mixture having a high proportion of n-pentanal from 2-butenes, i.e. to produce a product formed by terminal hydroformylation, it is necessary to use a catalyst which shifts the double bonds in the linear butenes under hydroformylation conditions, since n-pentanal can only be formed from 1-butene which is in equilibrium with the 2-butenes.

When a fast-isomerizing hydroformylation catalyst is used, an equilibrium between the double bond isomers is always established regardless of whether 1-butene, trans-2-butene, cis-2-butene or any mixture of linear butenes is chosen as starting material.

For this reason, a 2-butene/n-butane mixture as would have been able to be separated off from the reaction product mixture from Example 1 was not used in Example 2, but instead an industrially available stream (referred to as crude butane) having the following composition was used.

| | |
|---|---|
| n-Butane | 62% by mass |
| trans-2-Butene | 24% by mass |
| cis-2-Butene | 11% by mass |
| 1-Butene | 3% by mass |

The hydroformylation experiments were carried out in a 100 ml autoclave from Parr which was equipped with pressure maintenance device, flow meter, propellor stirrer and HPLC pump. A solution of Rh nonanoate (from Chempur) in toluene having an Rh nonanoate content of 0.0231 g and a solution of 0.2285 g of the ligand A (prepared by a method based on U.S. Pat. No. 4,885,401, UCC) in toluene were introduced into the autoclave under an argon atmosphere. The amount of toluene was then made up to 42 g.

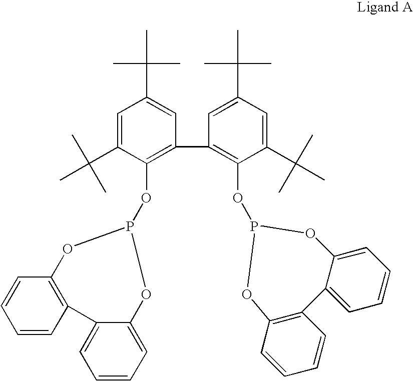

The rhodium/ligand mixture was subsequently brought to a pressure of 5-10 bar (intended pressure: 25 bar) by means of synthesis gas (CO: H₂ 1:1) while stirring (1000 rpm) and heated to 120° C. After the desired reaction temperature had been reached, 30 ml (18.8 g) of crude butane having the composition indicated above were metered in by means of an HPLC pump and the pressure was adjusted to the intended pressure of 25 bar. During the running time of the experiment, samples were taken at fixed time intervals. The reaction was carried out at a constant pressure (pressure regulator from Bronkhorst (NL)) for 5.5 hours. After the time of the experiment had elapsed, the autoclave was cooled to room temperature, vented and flushed with argon. 0.2 ml samples of the autoclave solution were in each case admixed with 0.8 ml of n-pentane and analyzed by gas chromatography.

| | Rh | Molar | | | | | Selectivity [%] |
|---|---|---|---|---|---|---|---|
| Example | concentration | ligand/Rh | Time | Temperature | Pressure | Conversion | a) to pentanals |
| No. | [ppm] | ratio | [h] | [° C.] | [bar] | [%] | b) to n-pentanal |
| 2a | 100 | 5/1 | 3.5 | 120 | 25 | 85.8 | a) 100 |
| | | | | | | | b) 82 |
| 2b | 100 | 5/1 | 5.5 | 120 | 25 | 95.3 | a) 100 |
| | | | | | | | b) 82 |

Example 2 shows that n-valeraldehyde can be produced with high n-selectivity and high conversions from the starting material containing only a small proportion of 1-butene.

The two Examples 1 and 2 demonstrate that a mixture of C₄-olefins can be converted substantially into products formed by terminal hydroformylation and, if applicable, a subsequent reaction.

The hydrogenation of aldehydes obtained by hydroformylation or of hydroformylation mixtures is general prior art. However, the hydroformylation mixtures obtained in Examples 1 and 2 were not hydrogenated and worked up to the give the corresponding pentanols but instead, in the interests of simplicity, industrially available pentanols (from Celanese) were used in the dehydration experiments (Examples 4-5).

### EXAMPLE 3

### Production of a Dehydration Catalyst

As support material for the production of a dehydration catalyst, use was made of an acid γ-aluminum oxide having an Na₂O content of less than 300 ppm by mass from Axens. This aluminum oxide having a BET surface area of 225 m²/g and a pore volume of 0.68 ml/g was in the form of extrudates (cylinders having a length of 4-6 mm and a diameter of 1.25 mm). As barium precursor for the basic modification of the aluminum oxide with barium oxide (BaO), used as made of barium nitrate Ba(NO₃)₂.

Before application of the barium salt, the aluminum oxide was firstly dried at 90° C. in a convection drying oven for 5 hours. 200 g of the dried extrudate were subsequently impregnated at room temperature with a solution consisting of 130 ml of water and 5.19 g of barium nitrate by means of a spray nozzle in a rotating drum (coating drum).

After impregnation, the extrudates laden with the barium salts were firstly dried at 110° C. in a convection drying oven for 5 hours. The subsequent calcination, in which the barium salt is converted into barium oxide or into a barium-aluminum-oxygen compound, was carried out in a stream of air at 450° C. in a fluidized-bed reactor for 10 hours. The finished catalyst contained 1.5% by mass of barium compounds, calculated as barium oxide.

### EXAMPLE 4

### Preparation of 1-pentene from 1-pentanol

1-Pentanol was reacted over the catalyst produced in Example 3 in an electrically heated fixed-bed flow reactor. Before entry into the reactor, the liquid starting material was vaporized at 220° C. in an upstream vaporizer. 24 g/h of 1-pentanol were passed in the gas phase through 15.1 g of catalyst, corresponding to a WHSV of 1.59 h⁻¹, at a reaction temperature of 350° C. The gaseous product was cooled in a condenser and collected in a glass receiver. The product had, on a water-free basis, the following composition determined by gas chromatography:

| | |
|---|---|
| 1-Pentene | 93.1% by mass |
| Trans-2-Pentene | 1.9% by mass |
| cis-2-Pentene | 4.1% by mass |
| 1-Pentanol | 0.1% by mass |
| Other components | 0.8% by mass |

### EXAMPLE 5

### Preparation of 3-methyl-1-butene from 3-methyl-1-butanol

3-Methyl-1-butanol was reacted over the catalyst produced in Example 3 in an electrically heated fixed-bed flow reactor. Before entry into the reactor, the liquid starting material was vaporized at 220° C. in an upstream vaporizer. 24 g/h of 3-methyl-1-butanol were passed in the gas phase through 15.1 g of catalyst, corresponding to a WHSV of 1.59 h⁻¹, at a reaction temperature of 340° C. The gaseous product was cooled in a condenser and collected in a glass receiver. The product had, on a water-free basis, the following composition determined by gas chromatography:

| | |
|---|---|
| 3-Methyl-1-butene | 91.1% by mass |
| cis-2-Methyl-2-butene | 4.5% by mass |
| trans-2-Methyl-2-butene | 2.5% by mass |
| 3-Methyl-1-butanol | 1.3% by mass |
| Other components | 0.6% by mass |

### EXAMPLE 6

### Production of a Rhenium/Aluminum Oxide Metathesis Catalyst

A mixture of 20 g of γ-aluminum oxide pellets, 1.39 g of rhenium heptoxide (Re₂O₇) (from Merck), 270 ml of 1,4-dioxane and 30 ml of water was refluxed for 16 hours. The mixture was evaporated to dryness by means of a rotary evaporator at temperatures up to 80° C. and a reduced pressure down to 20 mbar. The solid which remained was introduced into a metathesis reactor and predried in this at 120° C. in a stream of nitrogen (5 l/h) for one hour. The temperature was subsequently increased to 550° C. in the stream of nitrogen. After this temperature had been reached, the gas passed through the reactor was changed over to a gas mxiture consisting of 20% of oxygen and 80% of nitrogen while maintaining the same gas throughput. After 16 hours at 550° C., the catalyst was cooled to room temperature in a stream of nitrogen (5 l/h). The catalyst contained 6.5% by mass of rhenium oxide.

### Metathesis Experiments (Examples 7 to 9)

The starting material (pentenes) was conveyed by means of a peristaltic pump from a reservoir which was cooled to −15° C. and stood on a balance in order to quantify the mass used (or the mass flow) via a vaporizer (round-bottomed flask in an oil bath at 100° C.) to the top of a metathesis reactor. This reactor comprised a tube having a length of 500 mm and a diameter of 24 mm. The reactor was thermostated by means of jacket heating. The liquid products were condensed at −15° C. from the mixture leaving the reactor, weighed and analyzed by gas chromatography.

### EXAMPLE 7

### Metathesis of 1-pentene

1-Pentene was passed at a rate of 95 g/h over 21.4 g of rhenium oxide/aluminum oxide catalyst (Example 6) at a temperature of 100° C. The 1-pentene conversion was 35% and the selectivity to 4-octenes was 98.7%.

### EXAMPLE 8

### Metathesis of 3-methyl-1-butene

3-Methyl-1-butene was passed at a rate of 95 g/h over 21.4 g of rhenium oxide/aluminum oxide catalyst (Example 6) at a temperature of 100° C. The 3-methyl-1-butene conversion was 35% and the selectivity to 2,5-dimethyl-3-hexenes was over 99%.

### EXAMPLE 9

### Metathesis of a mixture of 1-pentene and 3-methyl-1-butene

An equimolar mixture of 1-pentene and 3-methyl-1-butene was passed at a rate of 95 g/h over 21.4 g of rhenium oxide/aluminum oxide catalyst (Example 6) at a temperature of 100° C. The conversion of the two starting olefins was 35% and the yield of C₈-olefins was 99%. The C₈-olefin fraction consisted of 25% of 4-octenes, 25% of 2,5-dimethyl-3-hexenes and 50% of 2-methyl-3-heptenes.

1. A process for preparing at least one olefin having from 8 to 12 carbon atoms from at least one olefin having from 4 to 6 carbon atoms by means of a four-stage synthesis, which comprises
a) hydroformylating at least one starting olefin in the first process step,
b) hydrogenating the at least one aldehyde obtained in the first step a) to form the corresponding alcohol,
c) preparing at least one 1-olefin by elimination of water from the at least one alcohol obtained in the second process step b) and
d) obtaining at least one olefin by metathesis with elimination of ethylene from the at least one 1-olefin(s) obtained in the third process step c).

2. The process as claimed in claim 1,
wherein a mixture of olefins having from 4 to 6 carbon atoms is used and a mixture of olefins having from 8 to 12 carbon atoms is obtained.

3. The process as claimed in claim 1,
wherein a nickel, copper, copper/nickel, copper/chromium, copper/chromium/nickel, zinc/chromium, nickel/molybdenum catalyst is used as catalyst in the second process step b).

4. The process as claimed in claim 1,
wherein the elimination of water in the third process step c) is carried out continuously over a solid catalyst which consists formally of aluminum oxide and barium oxide.

5. The process as claimed in claim 1,
wherein a rhenium catalyst comprising Re₂O₇ on γ-Al₂O₃ or on mixed supports selected from among SiO₂/Al₂O₃, B₂O₃/SiO₂/Al₂O₃ or Fe₂O₃/Al₂O₃ is used in the fourth process step d).

6. The process as claimed in claim 1,
wherein a hydrocarbon stream comprising or consisting of isobutene and linear butenes is used as starting material in process step a).

7. The process as claimed in claim 1,
wherein a C₄ fraction selected from among raffinate I, selectively hydrogenated C₄ fraction from a cracker, C₄ fractions from FCC plants or C₄-olefins prepared by the Fischer-Tropsch synthesis is used as starting material.

8. The process as claimed in claim 7,
wherein industrial C₄ fractions having an isobutene content of greater than 3% by weight are used as starting material.

9. The process as claimed in claim 6,
wherein 3-methyl-1-butene is separated off from the 1-olefin fraction comprising olefins having 5 carbon atoms which is obtained after the third process step c).

10. A mixture which comprises at least one olefin having from 8 to 12 carbon atoms and has been prepared by a process as claimed in claim 1.

11. Isooctene prepared by a process as claimed in claim 1 using a C₄ fraction having an isobutene content of greater than 3% by weight as starting material.

12. The method of using a mixture as claimed in claim 10 for preparing alcohols and/or aldehydes.

13. The method of using a mixture as claimed in claim 12 for preparing plasticizer alcohols.

14. The method of using a mixture as claimed in claim 12 for preparing isononanol.
